(19) United States
(12) Patent Application Publication
(10) Pub. No.: US 2006/0292250 A1
(43) Pub. Date: Dec. 28, 2006

(54) Supplement composition and method of use for enhancement of anti-inflammation process

(75) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(73) Assignee: Suracell, Inc., Montclair, NJ (US)

(21) Appl. No.: 11/439,642

(22) Filed: May 24, 2006

Related U.S. Application Data
(60) Provisional application No. 60/693,527, filed on Jun. 23, 2005.

(51) Int. Cl. A61K 36/906; A61K 36/28; A61K 36/185; A61K 31/737; A61K 31/7008

(52) U.S. Cl. 424/756; 424/764; 424/778; 514/54; 514/62

(57) ABSTRACT

A supplement composition for enhancement of anti-inflammation process is provided, which contains nettle leaf extract, hops extract, and turmeric rhizome extract. The supplement composition further includes hyaluronic acid, sodium salt, glucosamine sulfate, and chondroitin sulfate. Further provided is a method of using the supplement composition for enhancement of anti-inflammation process.

### CROSS REFERENCE TO RELATED APPLICATION

This application claims the benefit under 35 USC 119(e) of the provisional patent application Ser. No. 60/693,527, filed Jun. 23, 2005, which is herein incorporated by reference in its entirety.

### FIELD OF THE INVENTION

The present invention relates to dietary supplement compositions and the method of use for enhancement of the anti-inflammation process.

### BACKGROUND OF THE INVENTION

Turmeric has been used in various parts of the world to treat many conditions. Curcuminoids are polyphenolic pigments found in the spice turmeric. The term turmeric is used both for the plant Curcuma longa L. and the spice derived from the rhizomes of the plant. The curcuminoids have been found to have antioxidant activity, anticarcinogenic, anti-inflammatory, antiviral and hypocholesterolemic activities. Curcumin and the other curcuminoids have been entered into Phase I clinical trials for cancer chemoprevention by the National Cancer Institute. The anti-inflammatory properties of the curcuminoids have been demonstrated in a number of in vitro and in some animal studies. These substances are widely used in India and Indonesia for various inflammatory conditions. In vitro, curcumin inhibits the production of pro-inflammatory cytokines such as tumor necrosis factor-alpha (TNF-α), interleukin-1 beta (IL-1 β) and interleukin-8, among other anti-inflammatory actions. Some preliminary evidence that one or more constituents of turmeric may be helpful in some forms of arthritis has emerged from a few animal studies and some human work.

Stinging nettle (Urtica dioica and the closely related Urtica urens) has a long medicinal history; it has been used for hundreds of years to treat rheumatism, eczema, arthritis, gout, and anemia. Today, many people use it to treat urinary problems during the early stages of benign prostatic hyperplasia (BPH), for urinary tract infections, kidney stones, hay fever (allergic rhinitis), or in compresses or creams for treating joint pain, sprains and strains, tendonitis, and insect bites.

Hops (Humulus lupulus) is a plant with a stout root, heart-shaped leaves and cone-like flowers. Hops is useful as a sleep inducer, has antioxidant activity, anti-inflammatory, estrogen-like properties, and potential anti-tumor activity. Hops extract consists of the dried strobile of Humulus lupulus L. and their preparations. Hop strobile contains resinous bitter principles (5-30%), mostly a-bitter acids (humulones 2-10%) and b-bitter acids (lupulones 2-16%) and their oxidative degradation products (2-methyl-3-buten-2-ol); polyphenolic condensed tannins (2-4%); volatile oil (0.35-1.0%), mainly monoterpenes and sesquiterpenes (b-caryophyllene, farnesene, humulene, b-myrcene); chalcones (xanthohumol); flavonoids (kaempferol, quercetin, rutin); phenolic acids; and amino acids (Bradley, P. R. (ed.). 1992. British Herbal Compendium, Vol. 1. Bournemouth: British Herbal Medicine Association; Bruneton, J. 1995. Pharmacognosy, Phytochemistry, Medicinal Plants. Paris: Lavoisier Publishing; “Lupuli strobulus.” Monographs on the Medicinal Uses of Plant Drugs. Exeter, U.K.: European Scientific Cooperative on Phytotherapy; Leung, A. Y. and S. Foster. 1996. Encyclopedia of Common Natural Ingredients Used in Food, Drugs, and Cosmetics, 2nd ed. New York: John Wiley & Sons, Inc.; Newall, C. A., L. A. Anderson, J. D. Phillipson. 1996. Herbal Medicines: A Guide for Health-Care Professionals. London: The Pharmaceutical Press; Wichtl, M. and N. G. Bisset (eds.). 1994. Herbal Drugs and Phytopharmaceuticals. Stuttgart: Medpharm Scientific Publishers).

Hyaluronic acid is widely applied in health and beauty food in America, Japan and Europe. Hyaluronic acid is a mucopolysaccharide composed of repeat disaccharide units of N-acetylglucosamine and glucuronic acid. Hyaluronic acid is widely found in the extracellular matrix of animal tissues, vitreous humor, umbilical cord, skin, joints synovia and cockscomb etc. Its molecular weight is from several ten thousand to several million Daltons. Its main biological role is water retention and lubrication and osmoregulation.

Biochemically, glucosamine is involved in glycoprotein metabolism. Glycoproteins, known as proteoglycans, form the ground substance in the extra-cellular matrix of connective tissue. In the early stages of degenerative joint disease, aggrecan biosynthesis is increased. However, in later stages, aggrecan synthesis is decreased, leading eventually to the loss of cartilage resiliency and to most of the symptoms that accompany osteoarthritis. During the progression of osteoarthritis, exogenous glucosamine can have a beneficial role. It is also known that, in vitro, chondrocytes do synthesize more aggregan when the culture medium is supplemented with glucosamine. Glucosamine also has anti-inflammatory properties.

Chondroitin sulfate belongs to a family of heteropolysaccharides called glycosaminoglycans or GAGs. GAGs in the form of proteoglycans comprise the ground substance in the extracellular matrix of connective tissue. Chondroitin sulfate is made up of linear repeating units containing D-galactosamine and D-glucuronic acid. Chondroitin sulfate is found in humans in cartilage, bone, cornea, skin and the arterial wall. This type of chondroitin sulfate is sometimes referred to as chondroitin sulfate A or galactosaminoglucuronoglycan sulfate. The molecular weight of chondroitin sulfate ranges from 5,000 to 50,000 daltons and contains about 15 to 150 basic units of D-galactosamine and D-glucuronic acid. Chondroitin sulfate C, primarily found in fish and shark cartilage, but also in humans, is also made up of linear repeating units of D-galactosamine and D-glucuronic acid. Chondroitin sulfate supplements are usually isomeric mixtures of chondroitin sulfate A (chondroitin 4-sulfate) and chondroitin sulfate C (chondroitin 6-sulfate). Actions of orally administered chondroitin sulfate include promotion and maintenance of the structure and function of cartilage (referred to as chondroprotection), pain relief of osteoarthritic joints and anti-inflammatory activity.

It is desirable to provide improved dietary supplements to enhance an individual's anti-inflammatory activities, particularly to those who are prone to inflammation due to genetic predisposition, clinical conditions, or advanced aging.

### SUMMARY OF THE INVENTION

In one aspect, the present invention is directed to a supplement composition for enhancement of anti-inflammation process. In one embodiment, the supplement composition comprises effective amounts of nettle leaf extract, hops extract; and turmeric rhizome extract. In a further embodiment, the supplement composition further comprises effective amounts of hyaluronic acid sodium salt, glucosamine sulfate; and chondroitin sulfate. Moreover, the supplement composition also comprises pharmaceutically acceptable excipients, and can be provided in a form for oral administration.

In a further aspect, the present invention is directed to a method for enhancement of an individual's anti-inflammation process. In one embodiment, as a regular strength treatment, the method comprises orally administering a supplement composition to a person daily in a dosage comprising from about 250 mg to about 1,500 mg of nettle leaf extract, from about 125 mg to about 1,000 mg of hops extract, and from about 50 mg to about 600 mg of turmeric rhizome extract.

In another embodiment, as an extra strength treatment, the method comprises orally administering a supplement composition to a person daily in a dosage comprising from about 250 mg to about 1,500 mg of nettle leaf extract, from about 125 mg to about 1,000 mg of hops extract, and from about 50 mg to about 600 mg of turmeric rhizome extract, from about 5 mg to about 20 mg of hyaluronic acid sodium salt, from about 125 mg to about 500 mg of glucosamine sulfate, and from about 100 mg to about 400 mg of chondroitin sulfate.

### DETAILED DESCRIPTION OF THE INVENTION

In one embodiment, the present invention provides a supplement composition for enhancing an individual's anti-inflammatory activities, and reducing the likelihood of an individual in developing inflammatory diseases. The supplement composition comprises nettle leaf extract, hops extract, turmeric rhizome extract, and pharmaceutically acceptable excipients.

The turmeric rhizome extract used in the composition preferably contains about 95% curcuminoids. Curcuminoids are polyphenolic pigments found in the turmeric. Curcuminoids are derived from turmeric by extraction with ethanol. The major curcuminoids are curcumin, demethoxycurcumin and bisdemethoxycurcumin. Curcuminoids include about 70 to 75% of curcumin, about 15 to 20% of demethoxycurcumin, and about 3% of bisdemethoxycurcumin. Curcumin is the most studied of the curcuminoids. In pure form, it is an orange-yellow, crystalline powder that is insoluble in water. Its chemical name is (E,E)-1,7-bis(4-hydroxy-3-methoxyphenyl)-1,6-heptadiene-3, 5 dione, and its molecular weight is 368.39 daltons. Curcumin and the other curcuminoids have been found to have antioxidant and anti-inflammatory activities. The anti-inflammatory activity of the curcuminoids may be accounted for by several mechanisms, including inhibition of COX and LOX, reduction of the release of ROS by stimulated neutrophils, inhibition of AP-1 and NF-KappaB, and inhibition of the activation of the pro-inflammatory cytokines TNF-α and IL (interleukin)-1 beta. The turmeric standardized rhizome extract used in the supplement composition of the present invention contains about 95% curcuminoids. In one embodiment, the supplement composition comprises the turmeric standardized rhizome extract in an amount from about 50 mg to about 200 mg in one dosage. In a preferred embodiment, the supplement composition comprises about 100 mg of turmeric standardized rhizome extract in one dosage. In another preferred embodiment, the supplement composition comprises about 300 mg of turmeric standardized rhizome extract in one dosage.

Herein, one dosage is also referred to as one serving. If the supplement composition is provided in the form of tablet, one dosage can be either one tablet, or two to three tablets. For example, 100 mg of turmeric standardized rhizome extract in one dosage can be provided in one tablet as shown in Supplement Composition A of Example 1; and 300 mg of turmeric standardized rhizome extract in one dosage can be provided in two tablets as shown in Supplement Composition B of Example 2. The size and number of tablets may depend on the manufacturability, which may further depend on the properties of the components and the pharmaceutically acceptable excipients used.

The supplement composition of the present invention comprises nettle leaf extract in an amount from about 250 mg to about 1,500 mg in one dosage. In a preferred embodiment, the supplement composition comprises about 500 mg of nettle leaf extract in one dosage. In another preferred embodiment, the supplement composition comprises about 750 mg of nettle leaf extract in one dosage.

The supplement composition of the present invention comprises hops extract in the form of a commercial product under the tradename IsoOxygene™, which contains botanical COX 2 inhibitor from Humulus Lupulus L. It is noted that herein the terms of hops extract and hops flower extract have been used interchangeably. Preferably, the supplement composition comprises hops extract in an amount from about 125 mg to about 1,000 mg in one dosage. In a preferred embodiment, the supplement composition comprises about 250 mg of hops extract in one dosage. In another preferred embodiment, the supplement composition comprises about 500 mg of hops extract in one dosage.

Example 1 provides an exemplary supplement composition of the present invention, which is considered as a regular strength formula.

In a further embodiment, the supplement composition of the present invention further comprises hyaluronic acid sodium salt, glucosamine sulfate, and chondroitin sulfate for further enhancement of anti-inflammation process.

Hyaluronic acid is known as being produced in the highest level during embryonic period of life, gradually decreasing after birth. Hyaluronic acid content is different among the people of the same age. For example, hyaluronic acid content of the premature senile patient is substantially reduced. Oral hyaluronic acid can increase endogenetic hyaluronic acid by digesting and absorbing. Hyaluronic acid also plays an important role of water retention and lubrication in tissues and organs, such as joint cavity, blood vessel, heart and brain. Decrease of hyaluronic acid can lead to arthritis, arteriosclerosis, pulse disorder and brain atrophy. Oral hyaluronic acid can restore the normal content of hyaluronic acid in the body, delay apolexis and make the body energetic. The commercial hyaluronic acid is commonly a sodium salt, called sodium hyaluronate. In one embodiment, the supplement composition of the present invention comprises hyaluronic acid sodium salt, preferably in an amount from about 5 mg to about 20 mg in one dosage. In a preferred embodiment, the supplement composition comprises about 10 mg of hyaluronic acid sodium salt in one dosage.

Glucosamine is an amino monosaccharide found in chitin, glycoproteins and glycosaminoglycans. Glucosamine is also known as 2-amino-2-deoxyglucose, 2-amino-2-deoxy-beta-D-glucopyranose and chitosamine. The glucosamine used in supplements is typically derived from marine exoskeletons. Synthetic glucosamine is also available. Glucosamine is available commercially as a nutritional supplement in three forms: glucosamine chloride, glucosamine sulfate and N-acetyl-glucosamine. Preferably, glucosamine sulfate is used because of its clinical effectiveness for treating osteoarthritis. Glucosamine sulfate is stabilized with one of two mineral salts: sodium chloride or potassium chloride. Both salts can effectively stabilize glucosamine sulfate; the use of potassium chloride as a stabilizer is preferable, because it does not increase the daily intake of sodium. In one embodiment, the supplement composition of the present invention comprises glucosamine sulfate 2KCl, preferably in an amount from about 125 mg to about 500 mg in one dosage. In a preferred embodiment, the supplement composition comprises about 250 mg of glucosamine sulfate 2KCl in one dosage.

Biochemistry and physiology of chondroitin sulfate is known. The polysaccharides, which include chondroitin sulfate and hyaluronic acid, make up as much as 95% of the proteoglycan structure. Chondroitin sulfate and hyaluronic acid are fundamental components of aggrecan found in articular cartilage, and they are vital for the structure and function of articular cartilage. Animal studies have shown that parenterally administered chondroitin sulfate does get into cartilage tissue as does orally administered chondroitin sulfate. There is also human data suggesting orally administered chondroitin sulfate, particularly low-molecular-weight chondroitin sulfate, is also delivered to articular tissue. Furthermore, there is also indication that orally administered chondroitin sulfate leads to increases in hyaluronic acid and viscosity of synovial fluid, as well as decreases in collagenase in synovial fluid. In one embodiment, the supplement composition of the present invention comprises chondroitin sulfate, preferably in an amount from about 100 mg to about 400 mg in one dosage. In a preferred embodiment, the supplement composition comprises about 200 mg of chondroitin sulfate in one dosage.

Example 2 provides an exemplary supplement composition of the present invention, which is considered as an extra strength formula.

As a convenient form of dietary supplement, the supplement compositions described above are provided in the form of tablet. However, it should be understood that tablet is only one of various convenient dosage forms which can be used for the supplement composition. Other suitable forms include hard or soft-gelatin capsules, powders, or in liquid dosage forms, such as elixirs, syrups, dispersed powders or granules, emulsions, or aqueous or oily suspensions. When other dosage forms are used, the amounts of the active components in one dosage remain the same; however, the concentration of the component in different pharmaceutical media can be different.

Preferably, the supplement composition is formulated as a tablet, and as such it can contain pharmaceutically acceptable excipients, according to methods and procedures well known in the art. As used herein, “excipients” means substances that are of little or no therapeutic value, but useful in the manufacture and compounding of various pharmaceutical preparations, which form the medium of the supplement composition. These substances include coloring, flavoring, and diluting agents; emulsifying and suspending agents; ointment bases; pharmaceutical solvents; antioxidants and preservatives for the product; and miscellaneous agents. Suitable excipients are described in Remington's Pharmaceutical Sciences, Mack Publishing Company, a standard reference text in this field, which is incorporated herein by reference in its entirety.

As used herein, “tablets” are solid pharmaceutical dosage forms containing active ingredients with or without suitable diluents and prepared either by compression or molding methods well known in the art. Although tablets are most frequently discoid in shape, they may also be round, oval, oblong, cylindrical, or triangular. They may differ greatly in size and weight depending on the amount of active ingredients present and the intended method of administration. They are divided into two general classes, (1) compressed tablets, and (2) molded tablets or tablet triturates. In addition to the active ingredients, tablets contain a number of inert excipients or additives. A first group of such excipients includes those materials that help to impart satisfactory compression characteristics to the formulation, including diluents, binders, and lubricants. A second group of such excipients helps to give additional desirable physical characteristics to the finished tablet, such as disintegrators, colors, flavors, and sweetening agents. Compressed tablets can be uncoated or can be sugar coated or film coated by known techniques to mask any unpleasant taste and protect the tablet from the atmosphere, or enteric coated for selective disintegration and adsorption in the gastrointestinal tract.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and other suitable materials. As used herein, “binders” are agents used to impart cohesive qualities to the powdered material. Binders insure the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and other suitable materials. As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. As used herein, “coloring agents” are chemicals that give tablets a more pleasing appearance, and in addition help the manufacturer to control the product during its preparation and help the user to identify the product. Any of the approved certified water-soluble FD&C dyes, mixtures thereof, can be used to color tablets.

In a further aspect, the present invention provides the method of using the supplement compositions described above as a dietary supplement to individuals, particularly those in need thereof. Preferably, the supplement composition is administrated daily. The preferred daily dosage includes from about 250 mg to 1,500 mg of nettle leaf extract, from about 125 mg to about 1,000 mg of hops extract, and from about 50 mg to about 600 mg of turmeric rhizome extract. Furthermore, the daily dosage further includes from about 5 mg to about 20 mg of hyaluronic acid sodium salt, from about 125 mg to about 500 mg of glucosamine sulfate, and from about 100 mg to about 400 mg of chondroitin sulfate.

Supplement Composition A of Example 1 is considered as a regular strength formula for enhancing anti-inflammation process. The preferred daily dosage is one tablet of Supplement Composition A, which includes about 500 mg of nettle leaf extract, about 250 of hops extract, and about 100 mg of turmeric rhizome extract. Supplement Composition B of Example 2 is considered as an extra strength formula, which is particularly suitable for individuals who are prone to have inflammation due to genetic predisposition, clinical conditions, advanced aging, or poor environmental impact, and therefore, particularly in need of enhancement of anti-inflammation process. For these individuals, a preferred daily dosage is two tablets of Supplement Composition B, which has a daily dosage of about 750 mg of nettle leaf extract, about 300 mg of turmeric rhizome extract, about 500 mg of hops flower extract, about 10 mg of hyaluronic acid sodium salt, about 250 mg of glucosamine sulfate, and about 200 mg of chondroitin sulfate.

A method of determining an individual's genetic predisposition for deficiency in inflammation, in other words more prone to inflammation, is described in a co-pending patent application Ser. No. 60/796,423, entitled “Method of Determining Genetic Predisposition for Deficiency in Health Functions Using SNP Analysis”, which is herein incorporated by reference in its entirety. More specifically, upon performing a SNP genotyping assay of a biological sample collected from an individual, the individual's genetic predisposition for inflammation can be determined by using a specific inflammation SNP panel which comprises predetermined inflammation identifier SNPs. Such a SNP analysis determines and identifies an individual's genetic predisposition for inflammation as normal, sub-normal, and deficient. The individual whose genetic predisposition for inflammation is considered deficient is more likely to develop clinical conditions directly or indirectly related to inflammation.

In a further aspect, the method of the present invention provides a suitable supplement composition based on an individual's genetic predisposition for inflammation. In one embodiment, an individual whose genetic predisposition for inflammation is sub-normal is recommended to take one tablet of Supplement Composition A of Example 1 daily. In another embodiment of the present invention, an individual whose genetic predisposition for inflammation is deficient is recommended to take two tablets of Supplement Composition B of Example 2 daily. By providing a suitable antioxidant supplement composition based on an individual's genetic predisposition, the method of the present invention can more effectively assist in enhancing the individual's resistance to inflammation and reducing the likelihood of the individual in developing clinical conditions caused by inflammation.

The following examples are illustrative of the invention and are in no way to be interpreted as limiting the scope of the invention, as defined in the claims.

### EXAMPLE 1

A composition of the following formulation was prepared in caplet form, including the appropriate excipients, by standard methods known to those of ordinary skill in the art:

TABLE 1
Supplement Composition A

| | Amount |
|---|---|
| | Per Serving |
| Contents | (in one tablet) |
| Nettle Leaf Extract | 250 mg |
| Hops extract (in the form of IsoOxygene ™) | 125 mg |
| Turmeric standardized rhizome | 50 mg |
| extract (95% curcuminoids) | |

Other ingredients include: Dicalcium phosphate, microcrystalline cellulose, croscarmellose sodium, stearic acid, magnesium stearate, silica, and pharmaceutical glaze. In this example, each tablet has a weight ranging from about 1400 mg to about 1700 mg.

### EXAMPLE 2

A composition of the following formulation was prepared in caplet form, including the appropriate excipients, by standard methods known to those of ordinary skill in the art:

TABLE 2
Supplement Composition B

| | Amount Per Serving |
|---|---|
| Contents | (in two tablets) |
| Nettle Leaf Extract | 375 mg |
| Turmeric standardized rhizome | 150 mg |
| extract (95% curcuminoids) | |
| Hyaluronic acid, sodium salt | 5 mg |
| Glucosamine sulfate 2KCl | 125 mg |
| Chondroitin sulfate | 100 mg |
| Hops flower extract | 250 mg |

Other ingredients include: dicalcium phosphate, microcrystalline cellulose, croscarmellose sodium, stearic acid, magnesium stearate, silica, and pharmaceutical glaze. In this example, each tablet has a weight ranging from about 1400 mg to about 1700 mg.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A supplement composition for enhancement of anti-inflammation process, comprising effective amounts of:
(a) nettle leaf extract;
(b) hops extract; and
(c) turmeric rhizome extract.

2. The supplement composition of claim 1, wherein said composition comprises from about 250 mg to about 1,500 mg of said nettle leaf extract in one dosage.

3. The supplement composition of claim 1, wherein said composition comprises from about 125 mg to about 1,000 mg of said hops extract in one dosage.

4. The supplement composition of claim 1, wherein said composition comprises from about 50 mg to about 600 mg of said turmeric rhizome extract in one dosage.

5. The supplement composition of claim 1 further comprising effective amounts of.
(d) hyaluronic acid, sodium salt;
(e) glucosamine sulfate; and
(f) chondroitin sulfate.

6. The supplement composition of claim 5, wherein said composition comprises from about 5 mg to about 20 mg of said hyaluronic acid sodium salt in one dosage.

7. The supplement composition of claim 5, wherein said composition comprises from about 125 mg to about 500 mg of said glucosamine sulfate in one dosage.

8. The supplement composition of claim 5, wherein said composition comprises from about 100 mg to about 400 mg of said chondroitin sulfate in one dosage.

9. The supplement composition of claim 1 further comprising pharmaceutically acceptable excipients.

10. The supplement composition of claim 1, wherein said composition is in a form for oral administration.

11. A supplement composition for enhancement of anti-inflammation process, comprising in one dosage:
(a) from about 250 mg to 1,500 mg of nettle leaf extract;
(b) from about 125 mg to about 1,000 mg of hops extract; and
(c) from about 50 mg to about 600 mg of turmeric rhizome extract;

12. The supplement composition of claim 11 further comprising in one dosage:
(d) from about 5 mg to about 20 mg of hyaluronic acid sodium salt;
(e) from about 125 mg to about 500 mg of glucosamine sulfate; and
(f) from about 100 mg to about 400 mg of chondroitin sulfate.

13. The supplement composition of claim 11 further comprising pharmaceutically acceptable excipients.

14. The supplement composition of claim 11, wherein said composition is in a form for oral administration.

15. A method for enhancement of anti-inflammation process comprising orally administering a supplement composition to a person daily in a dosage comprising from about 250 mg to about 1,500 mg of nettle leaf extract; from about 125 mg to about 1,000 mg of hops extract; and from about 50 mg to about 600 mg of turmeric rhizome extract.

16. The method of claim 15, wherein said supplement composition further comprising in said dosage from about 5 mg to about 20 mg of hyaluronic acid sodium salt; from about 125 mg to about 500 mg of glucosamine sulfate; and from about 100 mg to about 400 mg of chondroitin sulfate.
